(19) United States
(12) Patent Application Publication
(10) Pub. No.: US 2015/0045366 A1
(43) Pub. Date: Feb. 12, 2015

(54) SPRAY DRIED FORMULATIONS

(71) Applicant: Gilead Sciences, Inc., Foster City, CA (US)

(72) Inventor: Joanna M. Koziara, Foster City, CA (US)

(73) Assignee: Gilead Sciences, Inc., Foster City, CA (US)

(21) Appl. No.: 14/381,921

(22) PCT Filed: Feb. 28, 2013
(86) PCT No.: PCT/US2013/028258

§ 371 Date: Aug. 28, 2014

Related U.S. Application Data
(60) Provisional application No. 61/605,341, filed on Mar. 1, 2012.

(51) Int. Cl. C07D 417/12; A61K 31/47; A61K 31/513; A61K 31/5377; A61K 31/675

(52) U.S. Cl. CPC C07D 417/12; A61K 31/5377; A61K 31/675; A61K 31/513; A61K 31/47
USPC 514/236.8

(57) ABSTRACT

The invention provides a spray dried formulation of Compound I: or a salt thereof as well as compositions comprising the spray dried formulations, and methods for making and using the spray dried formulations.

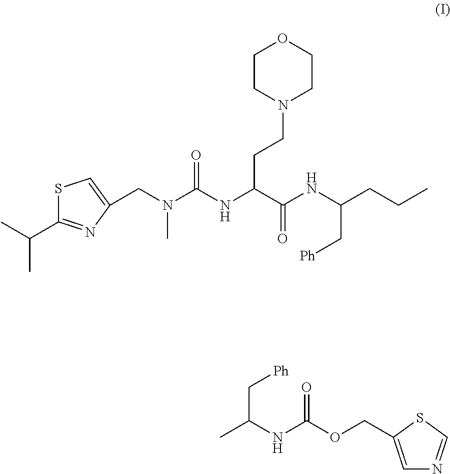

### CROSS REFERENCE TO RELATED APPLICATIONS

This patent application claims the benefit of priority of U.S. application Ser. No. 61/605,341, filed Mar. 1, 2012.

### BACKGROUND OF THE INVENTION

International patent application publication number WO 2008/010921 describes compounds and pharmaceutical compositions that improve the pharmacokinetics of a co-administered drug by inhibiting cytochrome P450 monooxygenase. One such inhibitor is the compound thiazol-5-ylmethyl (2R,5R)-5-((S)-2-(3-((2-isopropylthiazol-4-yl)methyl)-3-methylureido)-4-morpholinobutanamido)-1,6-diphenylhexan-2-ylcarbamate of formula (I).

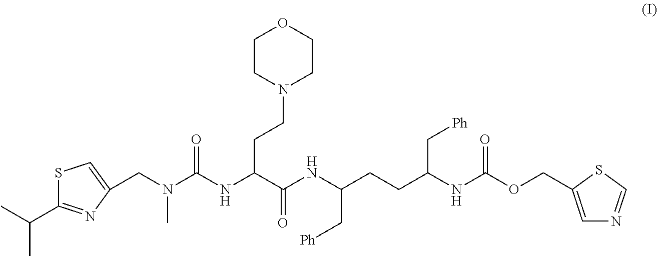 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

Unfortunately, the solid state properties of Formula I make it difficult to handle and process on a large scale. For example, its low glass transition temperature, hygroscopicity, and lack of crystallinity, as well as its non free-flowing nature make it particularly difficult to process and to formulate (e.g. as a tablet).

International patent application publication number WO 2009/135,179 discusses the difficulties associated with processing Formula I and describes combining Formula I with solid carrier particles to improve the physical properties of the resulting solid material. Accordingly, there is a need for improved solid forms of Formula I that have the beneficial properties of the solids described in WO 2009/135,179.

### SUMMARY OF THE INVENTION

The invention provides spray dried formulations of a compound of formula I that have many of the beneficial properties of the materials discussed in international patent application publication number WO 2009/135,179 and are amenable to formulation as a solid dosage form.

Accordingly, in one embodiment, the invention provides a formulation comprising Compound I:

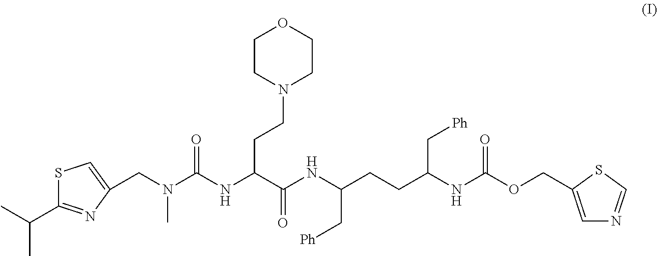 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

or a salt thereof, and a high glass transition temperature polymer.

In another embodiment, the invention provides a pharmaceutical composition comprising a formulation of the invention and a pharmaceutically acceptable excipient.

In another embodiment, the invention provides a tablet comprising a formulation of the invention.

In another embodiment, the invention provides a pharmaceutical composition comprising a formulation of the invention, and two or three additional therapeutic agents.

In another embodiment, the invention provides a pharmaceutical composition comprising a formulation of the invention, and two or three additional therapeutic agents wherein the two or three additional agents may be any of tenofovir disoproxil fumarate, emtricitabine and elvitegravir.

In another embodiment, the invention provides a method to inhibit the activity of cytochrome P-450 in an animal comprising administering a formulation of the invention to an animal.

In another embodiment, the invention provides a method for treating an HIV infection comprising administering to a patient in need thereof a therapeutically effective amount of a formulation of the invention, in combination with a therapeutically effective amount of one or more therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

In another embodiment, the invention provides a formulation of the invention for use in medical therapy.

In another embodiment, the invention provides a formulation of the invention for the prophylactic or therapeutic treatment of an HIV infection.

In another embodiment, the invention provides a formulation of the invention for use in the preparation of a medicament for treating HIV infection in a mammal.

In another embodiment, the invention provides a method for preparing a pharmaceutical composition comprising combining a formulation of the invention and a pharmaceutically acceptable excipient to provide the pharmaceutical composition.

In another embodiment, the invention provides a method for preparing a pharmaceutical composition comprising combining: i) a formulation of the invention, ii) tenofovir disoproxil fumarate, iii) emtricitabine, and iv) elvitegravir to provide the pharmaceutical composition.

### BRIEF DESCRIPTION OF THE DRAWINGS

FIG. 1. shows the results of dissolution experiments with Compound I from various spray dried samples at pH 7.0 in media containing 50 mM phosphate buffer pH 7.0 with 0.5% SLS using USP apparatus type II and paddle speed as follows: 0-60 min:50 rpm, 60-75 min:75 rpm, 75-90 min:100 rpm.

FIG. 2. shows the results of dissolution experiments with Compound I from various spray dried samples at pH 2 with media containing 0.01N HCL using USP apparatus type II with paddle speed as follows: 0-60 min:50 rpm, 60-75 min:100 rpm.

### DETAILED DESCRIPTION OF THE INVENTION

It will be appreciated by those skilled in the art that compounds of formula (I) may exist in and be isolated in optically active and racemic forms. Some compounds may exhibit polymorphism. It is to be understood that the present invention encompasses any racemic, optically-active, polymorphic, or stereoisomeric form, or mixtures thereof, of Compound I, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

A “spray dried formulation” as used herein includes solid materials prepared by spray drying a mixture comprising Compound (I) or a salt thereof, and a suitable polymer.

In one embodiment the invention provides pharmaceutical compositions comprising a formulation of the invention that can be administered to a mammalian host, such as a human patient, in a variety of forms adapted to the chosen route of administration (e.g. orally).

The compositions of the invention may include one or more pharmaceutically acceptable excipients. Excipients include but are not limited to substances that can serve as a vehicle or medium for a spray dried formulation of the invention (e.g. a diluent or a carrier). They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations will typically contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as hydroxypropyl cellulose, povidone, or hydroxypropyl methylcellulose; fillers, such as microcrystalline cellulose, pregelatinized starch, starch, mannitol, or lactose monohydrate; a disintegrating agent such as croscarmellose sodium, cross-linked povidone, or sodium starch glycolate; a lubricant such as magnesium stearate, stearic acid, or other metallic stearates; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, polymers, wax, shellac or sugar and the like. Of course, any material used in preparing any unit dosage form will typically be pharmaceutically acceptable and substantially non-toxic in the amounts employed. In addition, the compositions of the invention may be incorporated into sustained-release preparations and devices.

The compositions of the invention can also be administered topically, e.g., transdermally, buccally, or sublingually. Accordingly, the invention also provides pharmaceutical compositions that are formulated for such routes of topical administration. Useful dosages of the compounds of formula (I) can be determined by comparing their in vitro activity, and in vivo activity in animal models. Methods for the extrapolation of effective dosages in mice, and other animals, to humans are known to the art.

The amount of a composition of the invention required for use in treatment will vary with the route of administration, the nature of the condition being treated and the age and condition of the patient and will be ultimately at the discretion of the attendant physician or clinician.

In general, however, a suitable dose of Compound I will be in the range of from about 0.05 to about 100 mg/kg, e.g., from about 0.05 to about 50 mg/kg of body weight per day, preferably in the range of 0.05 to 10 mg/kg/day, most preferably in the range of 0.05 to 5 mg/kg/day.

The compound is conveniently formulated in unit dosage form; for example, containing about 5 to 500 mg, about 5 to 250 mg, or about 10 to 100 mg of Compound I. In one embodiment, the invention provides a composition comprising about 5, about 25, or about 100 mg of Compound I formulated in a unit dosage, and one or more pharmaceutically acceptable excipients.

The ability of Compound Ito inhibit cytochrome P-450 can be evaluated as described in international patent application publication number WO2008/010921.

### Combination Formulations

Compound I can be used to improve the pharmacokinetics of a co-administered drug, e.g., by inhibiting cytochrome P-450 monooxygenase. Accordingly, in another embodiment, the pharmaceutical compositions of the invention can further comprise at least one additional therapeutic agent, especially where the at least one additional therapeutic agent is metabolized by cytochrome P-450 monooxygenase.

The additional therapeutic agent can be any agent having a therapeutic effect when used in combination with the compound of the present invention. For example, the additional therapeutic agent used in combination with Compound I can be any agent that is accessible to oxidative metabolism by cytochrome P450 enzymes, especially cytochrome P450 monooxygenase, e.g., 1A2, 2B6, 2C8, 2C19, 2C9, 2D6, 2E1, 3A4, 5, 7, etc.

In one example, the additional therapeutic agent can be any anti-viral agent, e.g., anti-HIV, anti-HCV, etc., anti-bacterial agent, anti-fungal agent, immuno-modulator, e.g., immunosuppressant, anti-neoplastic agent, chemotherapeutic agent, agents useful for treating cardiovascular conditions, neurological conditions, etc. In another example, the additional therapeutic agent can be any proton pump inhibitor, anti-epileptics, NSAID, oral hypoglycemic agent, angiotensin II receptor antagonist, sulfonylurea, beta blocker, antidepressant, antipsychotic, or anesthetic, or a combination thereof.

In another example, the additional therapeutic agent can be any 1) macrolide antibiotic, e.g., clarithromycin, erythromycin, telithromycin, 2) anti-arrhythmic, e.g., quinidine=>3-OH, 3) benzodiazepine, e.g., alprazolam, diazepam=>3-OH, midazolam, triazolam, 4) immune modulator, e.g., cyclosporine, tacrolimus (FK506), 5) HIV antiviral, e.g., indinavir, nelfinavir, ritonavir, saquinavir, 6) prokinetic, e.g., cisapride, 7) antihistamine, e.g., astemizole, chlorpheniramine, terfenidine, 8) calcium channel blocker, e.g., amlodipine, diltiazem, felodipine, lercanidipine, nifedipine, nisoldipine, nitrendipine, verapamil, 9) HMG CoA reductase inhibitor, e.g., atorvastatin, cerivastatin, lovastatin, simvastatin, or 10) steroid 6beta-OH, e.g., estradiol, hydrocortisone, progesterone, testosterone.

In another example, the additional therapeutic agent can be alfentanyl, aprepitant, aripiprazole, buspirone, cafergot, caffeine, TMU, cilostazol, cocaine, codeine-N-demethylation, dapsone, dextromethorphan, docetaxel, domperidone, eplerenone, fentanyl, finasteride, gleevec, haloperidol, irinotecan, LAAM, lidocaine, methadone, nateglinide, ondansetron, pimozide, propranolol, quetiapine, quinine, salmeterol, sildenafil, sirolimus, tamoxifen, paclitaxel, terfenadine, trazodone, vincristine, zaleplon, or zolpidem or a combination thereof.

In one specific embodiment, the invention provides a pharmaceutical composition comprising, 1) a spray dried formulation of the invention, 2) at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, non-nucleoside inhibitors of HCV, CCR5 inhibitors, and combinations thereof, and 3) a pharmaceutically acceptable excipient.

In another embodiment, the present invention provides pharmaceutical compositions comprising 1) a spray dried formulation of the invention, 2) at least one additional therapeutic agent selected from the group consisting of amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, AG 1859, capravirine, emivirine, delaviridine, efavirenz, nevirapine, (+)-calanolide A, etravirine, GW5634, DPC-083, DPC-961, DPC-963, MIV-150, TMC-120, TMC-278 (rilpivirine), BILR 355 BS, VRX 840773, UK-453061, RDEA806, zidovudine, emtricitabine, didanosine, stavudine, zalcitabine, lamivudine, abacavir, amdoxovir, elvucitabine, alovudine, MTV-210, Racivir (±-FTC), D-d4FC, phosphazide, fozivudine tidoxil, apricitibine AVX754, amdoxovir, KP-1461, and fosalvudine tidoxil (formerly HDP 99.0003), tenofovir disoproxil fumarate, adefovir dipivoxil, GS-9131, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, L-870810, MK-0518 (raltegravir), elvitegravir, BMS-538158, GSK364735C, BMS-707035, MK-2048, BA 011, enfuvirtide, sifuvirtide, FB006M, TRI-1144, AMD-070, SP01A, BMS-488043, BlockAide/CR, immunitin, benzimidazole derivatives, benzo-1,2,4-thiadiazine derivatives, phenylalanine derivatives, aplaviroc, vicriviroc, and maraviroc, cyclosporine, FK-506, rapamycin, paclitaxel, taxotere, clarithromycin, A-77003, A-80987, MK-639, saquinavir, VX-478, AG1343, DMP-323, XM-450, BILA 2011 BS, BILA 1096 BS, BILA 2185 BS, BMS 186,318, LB71262, SC-52151, SC-629 (N,N-dimethylglycyl-N-(2-hydroxy-3-(((4-methoxyphenyl)sulphonyl)(2-methylpropyl)amino)-1-(phenylmethyl)propyl)-3-methyl-L-valinamide), KNI-272, CGP 53437, CGP 57813 and U-103017; and 3) a pharmaceutically acceptable excipient.

In another embodiment, the present invention provides pharmaceutical compositions comprising, 1) a spray dried formulation of the invention, and 2) two or three additional therapeutic agents. For example, additional therapeutic agents selected from the classes of HIV protease inhibitors, HIV non-nucleoside inhibitors of reverse transcriptase, HIV nucleoside inhibitors of reverse transcriptase, HIV nucleotide inhibitors of reverse transcriptase, and HIV integrase inhibitors. The two or three additional therapeutic agents can be different therapeutic agents selected from the same class of therapeutic agents, or they can be selected from different classes of therapeutic agents.

In another embodiment, the invention provides pharmaceutical compositions that comprise a ternary combination of agents selected from a spray dried formulation of the invention/tenofovir disoproxil fumarate/emtricitabine, a spray dried formulation of the invention/tenofovir disoproxil fumarate/elvitegravir, a spray dried formulation of the invention/tenofovir disoproxil fumarate/efavrenz, a spray dried formulation of the invention/tenofovir disoproxil fumarate/atazanavir, a spray dried formulation of the invention/tenofovir disoproxil fumarate/darunavir, a spray dried formulation of the invention/tenofovir disoproxil fumarate/raltegravir, a spray dried formulation of the invention/tenofovir disoproxil fumarate/rilpivirine, a spray dried formulation of the invention/GS-7340/emtricitabine, a spray dried formulation of the invention/GS-7340/elvitegravir, a spray dried formulation of the invention/GS-7340/efavrenz, a spray dried formulation of the invention/GS-7340/atazanavir, a spray dried formulation of the invention/GS-7340/darunavir, a spray dried formulation of the invention/GS-7340/raltegravir, a spray dried formulation of the invention/GS-7340/rilpivirine, a spray dried formulation of the invention/emtricitabine/elvitegravir, a spray dried formulation of the invention/emtricitabine/efavrenz, a spray dried formulation of the invention/emtricitabine/atazanavir, a spray dried formulation of the invention/emtricitabine/darunavir, a spray dried formulation of the invention/emtricitabine/raltegravir, a spray dried formulation of the invention/emtricitabine/rilpivirine, a spray dried formulation of the invention/elvitegravir/efavrenz, a spray dried formulation of the invention/elvitegravir/atazanavir, a spray dried formulation of the invention/elvitegravir/darunavir, a spray dried formulation of the invention/elvitegravir/raltegravir, a spray dried formulation of the invention/elvitegravir/rilpivirine, a spray dried formulation of the invention/efavrenz/atazanavir, a spray dried formulation of the invention/efavrenz/darunavir, a spray dried formulation of the invention/efavrenz/raltegravir, a spray dried formulation of the invention/efavrenz/rilpivirine, a spray dried formulation of the invention/a a zanavir/darunavir, a spray dried formulation of the invention/atazanavir/raltegravir, a spray dried formulation of the invention/atazanavir/rilpivirine, a spray dried formulation of the invention/darunavir/raltegravir, a spray dried formulation of the invention/darunavir/rilpivirine, and a spray dried formulation of the invention/raltegravir/rilpivirine.

In another embodiment, the invention provides pharmaceutical compositions that comprise a quaternary combination of agents selected from a spray dried formulation of the invention/tenofovir disoproxil fumarate/emtricitabine/elvitegravir, a spray dried formulation of the invention/tenofovir disoproxil fumarate/emtricitabine/efavrenz, a spray dried formulation of the invention/tenofovir disoproxil fumarate/emtricitabine/atazanavir, a spray dried formulation of the invention/tenofovir disoproxil fumarate/emtricitabine/darunavir, a spray dried formulation of the invention/tenofovir disoproxil fumarate/emtricitabine/raltegravir, a spray dried formulation of the invention/tenofovir disoproxil fumarate/emtricitabine/rilpivirine, a spray dried formulation of the invention/tenofovir disoproxil fumarate/elvitegravir/efavrenz, a spray dried formulation of the invention/tenofovir disoproxil fumarate/elvitegravir/atazanavir, a spray dried formulation of the invention/tenofovir disoproxil fumarate/elvitegravir/darunavir, a spray dried formulation of the invention/tenofovir disoproxil fumarate/elvitegravir/raltegravir, a spray dried formulation of the invention/tenofovir disoproxil fumarate/elvitegravir/rilpivirine, a spray dried formulation of the invention/tenofovir disoproxil fumarate/efavrenz/atazanavir, a spray dried formulation of the invention/tenofovir disoproxil fumarate/efavrenz/darunavir, a spray dried formulation of the invention/tenofovir disoproxil fumarate/efavrenz/raltegravir, a spray dried formulation of the invention/tenofovir disoproxil fumarate/efavrenz/rilpivirine, a spray dried formulation of the invention/tenofovir disoproxil fumarate/atazanavir/darunavir, a spray dried formulation of the invention/tenofovir disoproxil fumarate/atazanavir/raltegravir, a spray dried formulation of the invention/tenofovir disoproxil fumarate/atazanavir/rilpivirine, a spray dried formulation of the invention/tenofovir disoproxil fumarate/darunavir/raltegravir, a spray dried formulation of the invention/tenofovir disoproxil fumarate/darunavir/rilpivirine, a spray dried formulation of the invention/tenofovir disoproxil fumarate/raltegravir/rilpivirine, a spray dried formulation of the invention/GS-7340/emtricitabine/elvitegravir, a spray dried formulation of the invention/GS-7340/emtricitabine/efavrenz, a spray dried formulation of the invention/GS-7340/emtricitabine/atazanavir, a spray dried formulation of the invention/GS-7340/emtricitabine/darunavir, a spray dried formulation of the invention/GS-7340/emtricitabine/raltegravir, a spray dried formulation of the invention/GS-7340/emtricitabine/rilpivirine, a spray dried formulation of the invention/GS-7340/elvitegravir/efavrenz, a spray dried formulation of the invention/GS-7340/elvitegravir/atazanavir, a spray dried formulation of the invention/GS-7340/elvitegravir/darunavir, a spray dried formulation of the invention/GS-7340/elvitegravir/raltegravir, a spray dried formulation of the invention/GS-7340/elvitegravir/rilpivirine, a spray dried formulation of the invention/GS-7340/efavrenz/atazanavir, a spray dried formulation of the invention/GS-7340/efavrenz/darunavir, a spray dried formulation of the invention/GS-7340/efavrenz/raltegravir, a spray dried formulation of the invention/GS-7340/efavrenz/rilpivirine, a spray dried formulation of the invention/GS-7340/atazanavir/darunavir, a spray dried formulation of the invention/GS-7340/atazanavir/raltegravir, a spray dried formulation of the invention/GS-7340/atazanavir/rilpivirine, a spray dried formulation of the invention/GS-7340/darunavir/raltegravir, a spray dried formulation of the invention/GS-7340/darunavir/rilpivirine, a spray dried formulation of the invention/GS-7340/raltegravir/rilpivirine, a spray dried formulation of the invention/GS-9131/emtricitabine/elvitegravir, a spray dried formulation of the invention/emtricitabine/elvitegravir/efavrenz, a spray dried formulation of the invention/emtricitabine/elvitegravir/atazanavir, a spray dried formulation of the invention/emtricitabine/elvitegravir/darunavir, a spray dried formulation of the invention/emtricitabine/elvitegravir/raltegravir, a spray dried formulation of the invention/emtricitabine/elvitegravir/rilpivirine, a spray dried formulation of the invention/emtricitabine/efavrenz/atazanavir, a spray dried formulation of the invention/emtricitabine/efavrenz/darunavir, a spray dried formulation of the invention/emtricitabine/efavrenz/raltegravir, a spray dried formulation of the invention/emtricitabine/efavrenz/rilpivirine, a spray dried formulation of the invention/emtricitabine/atazanavir/darunavir, a spray dried formulation of the invention/emtricitabine/atazanavir/raltegravir, a spray dried formulation of the invention/emtricitabine/atazanavir/rilpivirine, a spray dried formulation of the invention/emtricitabine/darunavir/raltegravir, a spray dried formulation of the invention/emtricitabine/darunavir/rilpivirine, a spray dried formulation of the invention/emtricitabine/raltegravir/rilpivirine, a spray dried formulation of the invention/elvitegravir/efavrenz/atazanavir, a spray dried formulation of the invention/elvitegravir/efavrenz/darunavir, a spray dried formulation of the invention/elvitegravir/efavrenz/raltegravir, a spray dried formulation of the invention/elvitegravir/efavrenz/rilpivirine, a spray dried formulation of the invention/elvitegravir/atazanavir/darunavir, a spray dried formulation of the invention/elvitegravir/atazanavir/raltegravir, a spray dried formulation of the invention/elvitegravir/atazanavir/rilpivirine, a spray dried formulation of the invention/elvitegravir/darunavir/raltegravir, a spray dried formulation of the invention/elvitegravir/darunavir/rilpivirine, a spray dried formulation of the invention/elvitegravir/raltegravir/rilpivirine, a spray dried formulation of the invention/efavrenz/atazanavir/darunavir, a spray dried formulation of the invention/efavrenz/atazanavir/raltegravir, a spray dried formulation of the invention/efavrenz/atazanavir/rilpivirine, a spray dried formulation of the invention/efavrenz/darunavir/raltegravir, a spray dried formulation of the invention/efavrenz/darunavir/rilpivirine, a spray dried formulation of the invention/efavrenz/raltegravir/rilpivirine, a spray dried formulation of the invention/atazanavir/darunavir/raltegravir, a spray dried formulation of the invention/atazanavir/darunavir/rilpivirine, and a spray dried formulation of the invention/darunavir/raltegravir/rilpivirine.

### Combination Methods of Treatment

In one embodiment, the compositions of the invention that comprise a spray dried formulation of the invention can be used alone, e.g., for inhibiting cytochrome P450 monooxygenase. In another embodiment, the compositions of the invention can be used in combination with other active therapeutic ingredients or agents. Preferably, the other active therapeutic ingredients or agents are metabolized or accessible to the oxidative metabolism by cytochrome P450 enzymes, e.g., monooxygenase enzymes such as 1A2, 2B6, 2C8, 2C19, 2C9, 2D6, 2E1, 3A4, 5, 7, etc., thereby reducing the amount or rate at which the other active therapeutic agent or ingredient is metabolized, whereby the pharmacokinetics of the other active therapeutic agent or ingredient is improved. Such improvements can include elevating the blood plasma levels of the other therapeutic agent or ingredient or maintaining a more therapeutically effective blood plasma level of the other therapeutic active agent or ingredient compared to blood plasma levels of the other therapeutic agent or ingredient administered without the compositions of the invention that comprises a spray dried formulation of the invention.

Co-administration of a spray dried formulation of the invention with one or more other active therapeutic agents generally refers to simultaneous or sequential administration of the spray dried formulation of the invention and one or more other active therapeutic agents, such that therapeutically effective amounts of the spray dried formulation of the invention and one or more other active therapeutic agents are both present in the body of the patient.

Co-administration includes administration of unit dosages of the spray dried formulation of the invention before or after administration of unit dosages of one or more other active therapeutic agents, for example, administration of the spray dried formulation of the invention within seconds, minutes, or hours of the administration of one or more other active therapeutic agents. For example, a unit dose of a compound of a spray dried formulation of the invention can be administered first, followed within seconds or minutes by administration of a unit dose of one or more other active therapeutic agents. Alternatively, a unit dose of one or more other therapeutic agents can be administered first, followed by administration of a unit dose of spray dried formulation of the invention within seconds or minutes. In some cases, it may be desirable to administer a unit dose of a spray dried formulation of the invention first, followed, after a period of hours (e.g., 1 to 12 hours), by administration of a unit dose of one or more other active therapeutic agents. In other cases, it may be desirable to administer a unit dose of one or more other active therapeutic agents first, followed, after a period of hours (e.g., 1 to 12 hours), by administration of a unit dose of a spray dried formulation of the invention.

In yet another embodiment, the present invention provides a method for improving the pharmacokinetics of a drug which is metabolized by cytochrome P450 monooxygenase, comprising administering to a patient treated with said drug, a therapeutically effective amount of a composition of the invention that comprises a spray dried formulation of the invention.

In yet another embodiment, the present application provides a method for improving the pharmacokinetics of a drug which is metabolized by cytochrome P450 monooxygenase, comprising administering to a patient treated with said drug, a therapeutically effective amount of a composition of the invention that comprises a spray dried formulation of the invention.

In yet another embodiment, the present application provides a method for improving the pharmacokinetics of a drug which is metabolized by cytochrome P450 monooxygenase 3A, comprising administering to a patient treated with said drug, a composition of the invention that comprises a spray dried formulation of the invention.

In yet another embodiment, the present application provides a method for increasing blood plasma levels of a drug which is metabolized by cytochrome P450 monooxygenase, comprising administering to a patient treated with said drug, a composition of the invention that comprises a spray dried formulation of the invention.

In yet another embodiment, the present application provides a method for increasing blood plasma levels of a drug which is metabolized by cytochrome P450 monooxygenase 3A, comprising administering to a patient treated with said drug, a composition of the invention that comprises a spray dried formulation of the invention.

In yet another embodiment, the present application provides a method for inhibiting cytochrome P450 monooxygenase 3A in a patient comprising administering to a patient in need thereof an amount of a composition of the invention that comprises a spray dried formulation of the invention, effective to inhibit cytochrome P450 monooxygenase 3A.

In yet another embodiment, the present application provides a method for treating an HIV infection comprising administering to a patient in need thereof a therapeutically effective amount of a composition of the invention that comprises a spray dried formulation of the invention, in combination with a therapeutically effective amount of one or more additional therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

In yet another embodiment, the present application provides a method for treating an HIV infection comprising administering to a patient in need thereof a therapeutically effective amount of a composition of the invention that comprises a spray dried formulation of the invention, in combination with a therapeutically effective amount of one or more additional therapeutic agents selected from the group consisting of amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, and GW640385X, DG17, PPL-100, DG35, AG 1859, capravirine, emivirine, delaviridine, efavirenz, nevirapine, (+) calanolide A, etravirine, GW5634, DPC-083, DPC-961, DPC-963, MIV-150, TMC-120, TMC-278 (rilpivirine), efavirenz, BILR 355 BS, VRX 840773, UK-453061, RDEA806, zidovudine, emtricitabine, didanosine, stavudine, zalcitabine, lamivudine, abacavir, amdoxovir, elvucitabine, alovudine, MIV-210, racivir (±-FTC), D-d4FC, emtricitabine, phosphazide, fozivudine tidoxil, apricitibine (AVX754), amdoxovir, KP-1461, fosalvudine tidoxil (formerly HDP 99.0003), tenofovir disoproxil fumarate, adefovir dipivoxil, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, L-870810, MK-0518 (raltegravir), elvitegravir, BMS-538158, GSK364735C, BMS-707035, MK-2048, and BA 011, enfuvirtide, sifuvirtide, FB006M, and TRI-1144, AMD-070, an entry inhibitor, SP01A, BMS-488043, BlockAide/CR, a G6PD and NADH-oxidase inhibitor, immunitin, aplaviroc, vicriviroc, maraviroc, PRO-140, INCB15050, PF-232798 (Pfizer), CCR5 mAb004, BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDX010 (ipilimumab), PBS 119, ALG 889, and PA-1050040 (PA-040).

In yet another embodiment, the present application provides a method for treating an HCV infection comprising administering to a patient in need thereof a therapeutically effective amount of a composition of the invention that comprises a spray dried formulation of the invention, in combination with a therapeutically effective amount of one or more additional therapeutic agents selected from the group consisting of pegylated rIFN-alpha 2b, pegylated rIFN-alpha 2a, rIFN-alpha 2b, rIFN-alpha 2a, consensus IFN alpha (infergen), reaferon, intermax alpha, τ-IFN-beta, infergen+actimmune, IFN-omega with DUROS, locteron, albuferon, rebif, Oral interferon alpha, IFNalpha-2b XL, AVI-005, PEG-Infergen, and pegylated IFN-beta, rebetol, copegus, viramidine (taribavirin), NM-283, valopicitabine, R1626, PSI-6130 (R1656), HCV-796, BILB 1941, XTL-2125, MK-0608, NM-107, R7128 (R4048), VCH-759, PF-868554, GSK625433, SCH-503034 (SCH-7), VX-950 (telaprevir), BILN-2065, BMS-605339, ITMN-191, MX-3253 (celgosivir), UT-231B, IDN-6556, ME 3738, LB-84451, MitoQ, benzimidazole derivatives, benzo-1,2,4-thiadiazine derivatives, phenylalanine derivatives, A-831, A-689, zadaxin, nitazoxanide (alinea), BIVN-401 (virostat), PYN-17 (altirex), KPE02003002, actilon (CPG-10101), KRN-7000, civacir, GI-5005, ANA-975, XTL-6865, ANA 971, NOV-205, tarvacin, EHC-18, NIM811, DEBIO-025, VGX-410C, EMZ-702, AVI 4065, Bavituximab, Oglufanide, and VX-497 (merimepodib).

### Specific Embodiments of the Invention

Specific embodiments identified herein are for illustration; they do not in any way exclude other embodiments of the invention.

In one embodiment, the formulation of the invention is a spray dried formulation.

In one embodiment, the high glass transition temperature polymer has a glass transition temperature of at least about 100° C.

In one embodiment, the high glass transition temperature polymer is selected from the group consisting of HPMC E5, PVP, PVP-VA, HMPC-P AND HPMC-AS or mixtures of thereof.

In one embodiment, the high glass transition temperature polymer is HPMC E5.

In one embodiment, the salt of Compound I is a salt selected from the group consisting of ascorbate, benzoate, besylate, bromide, camphorosulfonate, chloride, citrate, dichloroacetate, edisylate, ethanesulfonate, fumarate, gentisate, hippurate, hydrochloride, ketoglutarate, lactate, maleate, malonate, naphtalenesulfate, nicotinate, oxalate, phosphate, saccharinate, succinate, sulfate, tartarate, tosylate, and xinafoate salts.

In one embodiment, the salt of Compound I is a phosphate salt.

In one embodiment, the formulation of the invention further comprises an excipient wherein the excipient is silicon dioxide.

In one embodiment, the spray dried formulation has a glass transition temperature of at least about 40° C.

In another embodiment, the spray dried formulation has a glass transition temperature of at least about 50° C.

In another embodiment, the spray dried formulation has a glass transition temperature of at least about 60° C.

In another embodiment, the spray dried formulation has a glass transition temperature of at least about 70° C.

In another embodiment, the spray dried formulation has a glass transition temperature of at least about 80° C.

In another embodiment, Compound I is a compound of formula (Ia):

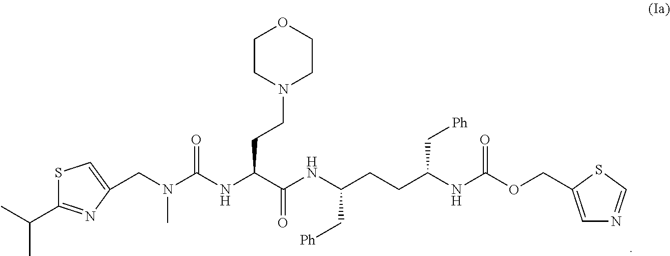 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

In one embodiment of the invention, Compound I is enriched with a stereoisomer of formula (I) that is formula (Ia):

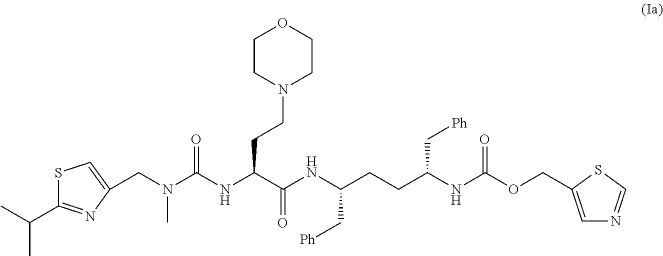 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

which is (3R,6R,9S)-12-methyl-13-[2-(1-methylethyl)-4-thiazolyl]-[2-(4-morpholinyl)ethyl]-8,11-dioxo-3,6-bis(phenylmethyl)-2,7,10,12-tetraazatridecanoic acid, 5-thiazolylmethyl ester. In one embodiment Compound I has an enriched concentration of 85±5% of the stereoisomer of formula (Ia). In another embodiment Compound I has an enriched concentration of 90±5% of the stereoisomer of formula (Ia). In another embodiment Compound I has an enriched concentration of 95±2% of the stereoisomer of formula (Ia). In another embodiment Compound I has an enriched concentration of 99±1% of the stereoisomer of formula (Ia). In another embodiment Compound I is the pure the stereoisomer of formula (Ia).

The invention will now be illustrated by the following non-limiting Examples.

### EXAMPLES

### 1. Preparation of Representative Spray Dried Compositions of the Invention (1)

Approximately 10 g of solids (compound I or salts of compound I and polymers) were dissolved in an appropriate solvent mixture. Alternatively additional excipients, for example silicon dioxide were added. Solution or suspensions were spray dried using an SD micro spray dryer (GEA-Niro SDMicro™ Spray Dryer, GEA Process Engineering, Columbia, Md.). Representative compositions of the spray drying feed solutions are summarized in Table 1.

TABLE 1
Summary of the Representative Spray Dried Solutions

| | | | | | | Final Solids |
|---|---|---|---|---|---|---|
| | | | Additional | Polymer: | | Concentration |
| Run | Polymer | API | excipients | API ratio | Solvent | (% w/w) |
| #1 | HPMC-P | Compound I | NA | 1:1 | 11% | 10 |
| | | free base | | | methanol in | |
| | | | | | acetone | |
| #2 | HPMC- | Compound I | NA | 1:1 | 1:1 | 7 |
| | E5 | Phosphate | | | methanol: | |
| | | salt | | | acetone | |
| #3 | PVP/VA | Compound I | NA | 1:1 | acetone | 20 |
| | | Xinafoate | | | | |
| | | salt | | | | |
| #4 | PVP | Compound I | 5% SiO2 | 1:1 | 1:1 ethanol: | 7 |
| | | phosphate | | | acetone | |
| | | salt | | | | |
| #5 | PVP | Compound I | 20% SiO2 | 1:1 | 1:1 ethanol: | 7 |
| | | phosphate | | | acetone | |
| | | salt | | | | |

### 2. Preparation of Representative Spray Dried Compositions of the Invention (2)

Spray drying feed solutions were prepared by dissolving compound I in free base form in 10% methanol in water or 1:1 DCM:methanol. Phosphate salt was formed in situ by addition of phosphoric acid into the solution, followed by addition of HPMC-E5. The ratio of phosphate salt and polymer were held constant at 1:1. Molar ratios of phosphoric acid to the compound I free base were 0.8, 1.0 and 1.2.

TABLE 2
Summary of the Representative Spray Dried
Solutions (In situ salt formation)

| | | | |
|---|---|---|---|
| | Acid (mol | Polymer:Compound I | |
| Run | equivalent) | phosphate ratio | solvent |

85% phosphoric acid

| | | | |
|---|---|---|---|
| #6 | 1.0 | 1:1 | 1:1 DCM:methanol |
| #7 | 1.2 | 1:1 | 1:1 DCM:methanol |
| #8 | 0.8 | 1:1 | 1:1 DCM:methanol |

Anhydrous phosphoric acid

| | | | |
|---|---|---|---|
| #9 | 1.0 | 1:1 | 10% water in methanol |
| #10 | 1.0 | 1:1 | 1:1 DCM:methanol |

### 3. Determination of Thermal Properties and Hygroscopicity

Modulated differential scanning calorimetry (mDSC) was used to characterize composition of the invention. Solid samples were placed in hermetically sealed aluminum pan with a pinhole. Modulation amplitude off 0.8° C. with a period of 60 sec was applied to the sample heated at 2° C./min under dried nitrogen purge using TA instruments (New Castle, Del., USA) model 1000.

Hygroscopicity of the compositions of invention were measured by placing approximately 20 to 50 mg of the sample in an uncapped scintillation vial. The vial was stored at ambient temperature in a Pyrex brand desiccator (VWR, International, West Chester, Pa.). The humidity within the desiccator was controlled at 55% relative humidity and 75% relative humidity using saturated aqueous solutions of magnesium nitrite and sodium chloride, respectively. The relative humidity was confirmed using a digital hygrometer pen (VWR International, West Chester, Pa.). The weight gain for all the samples was determined after 24 hours; it was not determined whether equilibrium was reached after that period. Visual observation of moisture induced phase transition was also noted.

Hygroscopicity and glass transition temperature for selected compositions of the invention are shown in Table 3 below.

TABLE 3
Summary of the Hygroscopicity and Glass Transition Temperature
of the Selected Compositions of the Invention

| | | Hygroscopicity | | | |
|---|---|---|---|---|---|
| | | Weight | | | |
| | | gain at | Phase | Weight gain | Phase |
| | | 55% RH | transi- | at 75% RH | transi- |
| Run # | Tg (° C.) | (%) | tion | (%) | tion |
| NA | 28, 49 | 1.9 | yes | 3.7 | yes |
| Compound | | | | | |
| Free base | | | | | |
| #1 | 79 | 1.4 | no | 4.3 | no |
| #2 | 75 | 4.5 | no | 8.4 | no |
| #3 | 76 | 2.4 | no | 7 | no |
| #4 | 73 | 9.9 | no | 17.8 | partial |
| #5 | 93 | 8.3 | no | 14.7 | no |
| #6 | 75.8 | 4.8 | no | 8.1 | no |
| #7 | 74.5 | 4.9 | no | 8.1 | no |
| #8 | 78.4 | 4.9 | no | 8.2 | no |
| #9 | 78.0 | 4.5 | no | 8.6 | no |
| #10 | 76.3 | 4.5 | no | 7.8 | no |

### 4. Particle Size Measurements

Particle size analysis was performed using Mastersizer 2000 (Malvern Instruments, Worcestershire, UK). Approximately 50 mg of sample was suspended in 20 ml of 2.5% Span 85 in mineral spirits (w/v) by sonication (10 to 20 seconds). Sample was then diluted to appropriate intensity in the same dispersant and particle size was measured. Data is reported as d(50), d(90) and volume weighted mean particle size in Table 4.

TABLE 4
Summery of the Particle Size and Density Data
for Selected Compositions of the Invention

| | | | | Bulk | Tapped |
|---|---|---|---|---|---|
| | Size (μm) | | | density | density |
| Solid composition | d(50) | d(90) | mean | (g/mL) | (g/mL) |
| Compound I phosphate | 24.1 | 62.5 | 30 | 0.3 | 0.47 |
| salt:HPMC E5 (2:1) | | | | | |
| Compound I phosphate | 17.2 | 45.7 | 21 | 0.24 | 0.37 |
| salt:HPMC E5 (3:1) | | | | | |

### 5. Dissolution Testing

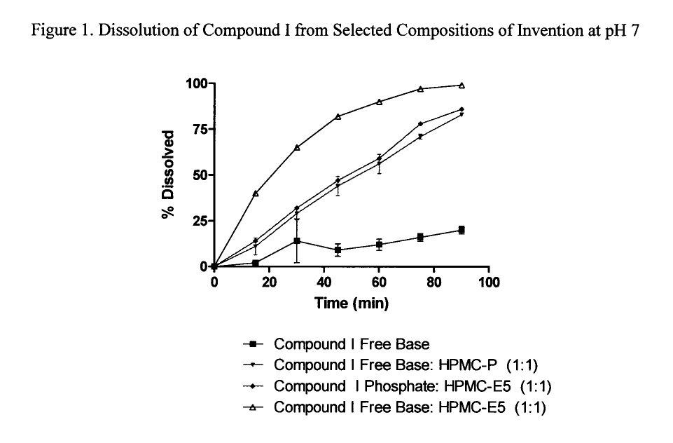

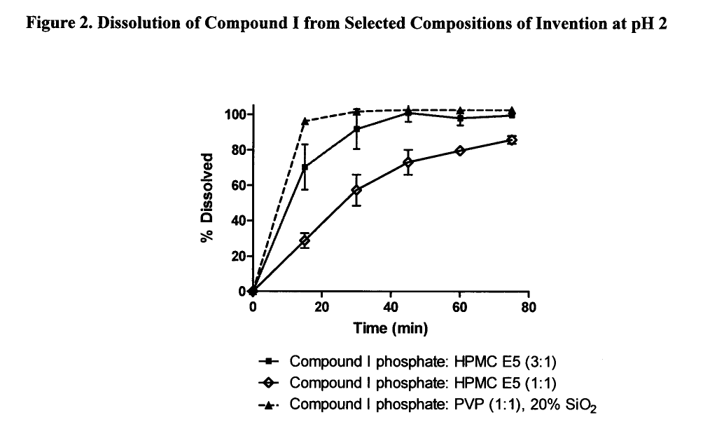

Dissolution testing was performed on the composition of the invention and Compound I (free base) using a USP paddle apparatus at 50 rpm (0 to 60 min), 75 rpm (60 to 75 min) and 100 rpm (75 to 90 min) with 500 mL of various media maintained at 37° C. Tested media included: 0.01N HCL (pH 2) and phosphate buffer pH 7.0 with 0.5% SLS. Spray dried powder was manually filled into hard gelatin capsules, which were weighed down with a sinker during dissolution testing. The extent of Compound I released as a function of time was monitored by HPLC using external reference standards at a wavelength of 240 nm. Results are shown in FIGS. 1 and 2.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A formulation comprising Compound I: 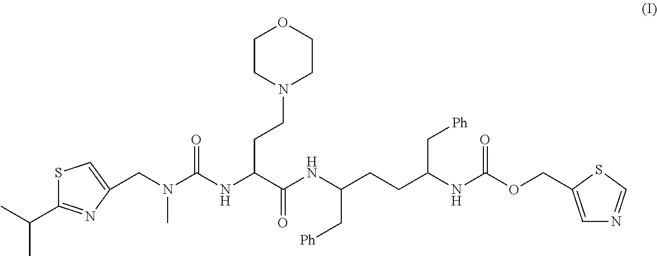 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]
or a salt thereof, and a high glass transition temperature polymer.

2. The formulation of claim 1 which comprises HPMC E5, PVP, PVP-VA, HMPC-P or HPMC-AS, or a mixture thereof.

3. The formulation of claim 2 wherein the high glass transition temperature polymer is HPMC E5.

4. The formulation of claim 1 wherein the salt of Compound I is a salt selected from the group consisting of ascorbate, benzoate, besylate, bromide, camphorosulfonate, chloride, citrate, dichloroacetate, edisylate, ethanesulfonate, fumarate, gentisate, hippurate, hydrochloride, ketoglutarate, lactate, maleate, malonate, naphtalenesulfate, nicotinate, oxalate, phosphate, saccharinate, succinate, sulfate, tartarate, tosylate, and xinafoate salts.

5. The formulation of claim 4 wherein the salt of Compound I is a phosphate salt.

6. The formulation of claim 1, wherein the formulation further comprises silicon dioxide.

7. The formulation of claim 1 that has a glass transition temperature of at least about 40° C.

8-9. (canceled)

10. The formulation of claim 9 that has a glass transition temperature of at least about 70° C.

11. The formulation of claim 10 that has a glass transition temperature of at least about 80° C.

12. The formulation of claim 1 wherein Compound I is a compound of formula (Ia):

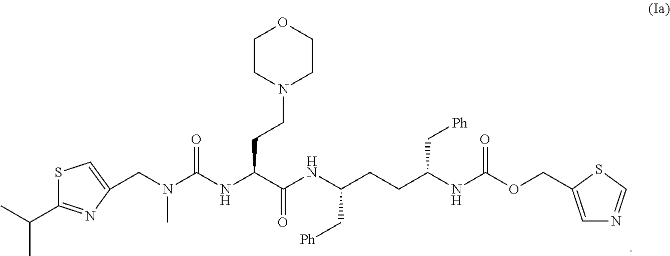 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

13. A pharmaceutical composition comprising a formulation of claim 1 and two or three additional therapeutic agents.

14. The pharmaceutical composition of claim 13 wherein the two or three additional agents are selected from the group consisting of tenofovir disoproxil fumarate, emtricitabine and elvitegravir.

15. A pharmaceutical composition comprising a formulation as described in claim 1 and a pharmaceutically acceptable excipient.

16. A tablet comprising a formulation or a composition as described in claim 1.

17. A method to inhibit the activity of cytochrome P-450 in an animal comprising administering a formulation as described in claim 1 to the animal.

18. A method for treating an HIV infection comprising administering to a patient in need thereof a therapeutically effective amount of a formulation as described in claim 1, in combination with a therapeutically effective amount of one or more therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

19. A formulation or composition as described in claim 1 for use in medical therapy.

20. The use of a formulation as described in claim 1 for the prophylactic or therapeutic treatment of an HIV infection.

21. (canceled)

22. A method for preparing a pharmaceutical composition comprising combining the formulation as described in claim 1 and a pharmaceutically acceptable excipient to provide the pharmaceutical composition.

23. A method for preparing a pharmaceutical composition comprising: combining a formulation as described in claim 1, tenofovir disoproxil fumarate, emtricitabine, and elvitegravir to provide the pharmaceutical composition.
